(19) United States
(12) Patent Application Publication
(10) Pub. No.: US 2010/0298872 A1
(43) Pub. Date: Nov. 25, 2010

(54) SURGICAL SUTURE MATERIAL CONSISTING OF BRAIDED THREAD

(75) Inventors: Ingo Berndt, Tuttlingen/Donau (DE); Erich Odermatt, Schaffhausen (CH)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: AESCULAP AG, Tuttlingen (DE)

(21) Appl. No.: 12/744,511

(22) PCT Filed: Nov. 26, 2008
(86) PCT No.: PCT/EP2008/010006

§ 371 Date: Aug. 12, 2010

(30) Foreign Application Priority Data
Nov. 26, 2007 (DE) 10 2007 058 256.2

(51) Int. Cl. A61B 17/04; D04C 1/00

(52) U.S. Cl. 606/228; 87/8

(57) ABSTRACT

A surgical suture includes a filamentary braid including filamentary elements (16; 20; 216; 220a-e; 34; 36; 44) projecting from the filamentary braid for anchoring in biological tissues.

### RELATED APPLICATIONS

This is a §371 of International Application No. PCT/EP2008/010006, with an inter-national filing date of Nov. 26, 2008 (WO 2009/068252 A1, published Jun. 4, 2009), which is based on German Patent Application No. 10 2007 058 256.2, filed Nov. 26, 2007, the subject matter of which is incorporated by reference.

### TECHNICAL FIELD

This disclosure relates to a surgical suture in the form of a filamentary braid, to a process for producing it, to a kit and also to methods of using the suture.

### BACKGROUND

The standard way of closing wounds in surgery is by using filamentary sutures. These are typically knotted to ensure secure anchoring in the tissues to be closed. The knotting of surgical suture here has a crucial influence on the quality of the wound closure, since it is not just the physical-chemical properties of sutures which decide the quality but also a correct knotting technique on the part of the surgeon.

Learning the correct knotting technique is a demanding and, more particularly, laborious exercise. Yet learning this technique is frequently not given the appropriate time and attention in the training of surgeons today. Therefore, the knotting of surgical suture is a frequent cause for the appearance of so-called “wound dehiscence.” The main sources of error here are more particularly an incorrect direction of knot throw, an incorrect choice of knot, knots tightened too little or too much, and also a positioning of the individual throws which is not appropriate to the wound medium. In addition, it is often necessary for multiple knots, more particularly up to 7 knots, to be placed on top of each other to ensure a secure knot hold. This represents a high input of material into the tissue and can lead to increased foreign-body reactions.

Therefore, suture research has for quite some time focused on knotless or self-fixing sutures. The knotless or self-fixing sutures which have become known to date include barbed sutures, also known as self-locking sutures or self-retaining sutures. They usually consist of a monofilament which includes barbs along its longitudinal axis. These barbs are usually created by cutting into the filamentary material. As a result, the filaments can be pulled through the tissue in the direction of the barbs. On pulling in the opposite direction, the barbs deploy and anchor themselves and hence the suture in the tissue by each barb drilling its own small, inclined puncture channel. This makes it impossible for the suture to be pulled back out of the puncture channel. These barbed sutures are well-known from the prior art. A suture of this kind is apparent from WO 2004/030520 A2. However, one disadvantage is the monofilamentary structure which normally underlies these sutures. Monofilaments are generally rather stiff structures and therefore unwieldy to handle. Moreover, the barbs cut into the monofilament constitute weak points in the suture, which reduce the mechanical strength of the latter. It is known from the technical literature that the barbs can lead to a reduction in linear tensile strength (LTS) by one United States Pharmacopeia (USP) size (R. Rashid, Arch. Dermatol. 2007, 143 (7), 869-872).

An example of a braided suture having barbs which project from a filamentary braid is derivable from US 2007/0005110 A2. In this suture, unbarbed filaments and at least one barbed filament are braidingly interlaced such that the unbarbed filaments enclose the barbed filament with the barbs extending outwardly from the enclosure. However, this does not cure the weakening of the surrounded barbed filament.

It would therefore be helpful to provide a surgical suture which permits knotless closure and/or knotless fixation of biological tissues.

### SUMMARY

We provide a surgical suture including a filamentary braid including filamentary elements projecting from the filamentary braid for anchoring in biological tissues.

We also provide a process for producing the suture, including producing the filamentary braid by braidingly intertwining filaments to form a braided structure, and forming filamentary elements projecting from the braided structure by leading individual filaments during braiding out of the braided structure which forms.

We further provide a surgical kit including the suture and at least a surgical needle.

### BRIEF DESCRIPTION OF THE DRAWINGS

The figures are schematic and show:

FIGS. 1a-1e and 2: a suture with various protruding filamentary elements;

FIGS. 3 and 4: a suture with tripled filaments;

FIG. 5: a suture with oppositely oriented, protruding filamentary ends;

FIG. 6: a scanning electron micrograph of a suture; and

FIG. 7: a scanning electron micrograph of a suture.

### DETAILED DESCRIPTION

We provide a surgical suture in the form of a filamentary braid wherein the suture includes filamentary elements projecting from the filamentary braid for anchoring in biological tissues.

We provide a surgical multifil suture whose projecting (protruding) filamentary elements allow knotless fixation or anchoring of the suture in a biological tissue. After implantation, a pulling load on the suture in the direction opposite to its direction of insertion will cause the filamentary elements to deploy and thereby anchor the suture in the tissue by boring their way into the tissue adjacent to the main puncture channel. The protruding filamentary elements here advantageously act as retaining or anchoring structures. This makes it possible to avoid weakening the substance of the suture, like in the case of the production of barbs in particular, since there are generally sufficient numbers present of continuous filamentary portions of other filaments that can absorb the pulling forces. In addition, the direction of the projecting filamentary elements can be subsequently adjusted and hence individually tailored.

Preferably, the projecting filamentary elements come from filaments of the filamentary braid and/or from filaments of a core of the filamentary braid. More preferably, the filamentary elements comprise ends of filaments, more particularly of the filamentary braid and/or of a core of the filamentary braid, and/or filamentary loops. The filamentary loops may comprise overfeeds, floats and/or velour loops. Filamentary loops have the advantage that they offer less resistance as the suture is inserted into a tissue, and hence minimize the risk of tissue traumatization.

Preferably, the projecting filamentary elements comprise opened filamentary loops, more particularly opened floats, opened overfeeds and/or opened velour loops. For example, the projecting filamentary elements may comprise severed filamentary loops.

The projecting filamentary elements are preferably present in a reinforced, more particularly stiffened, state. The filamentary elements may be in a physically, for example mechanically, or chemically reinforced state. The filamentary elements can be reinforced by polymers in particular. To this end, the filamentary elements can be dipped for example in liquid polymers or in polymer solutions, in which case the polymers are subsequently cured or hardened. The filamentary elements may further be in a thermally fixed state. The filamentary elements may also be in a welded, preferably ultrawelded, state.

The filamentary elements may in principle be formed in different arrangements on the filamentary braid. The filamentary elements may form a row-shaped arrangement, staggered arrangement, zigzag-shaped arrangement, spiral-shaped arrangement, random arrangement or combinations thereof on the filamentary braid. Preferably, the projecting filamentary elements form a regular distribution on the filamentary braid. For example, the projecting filamentary elements may be arranged in succession in the form of at least one row, more particularly one, two, three or more rows, preferably in the longitudinal direction of the filamentary braid.

Furthermore, the filamentary braid may have regions on its surface which are free of filamentary elements projecting from the braided structure. More particularly, surface regions displaying filamentary elements can alternate on the surface of the braided structure with surface regions displaying no filamentary elements. Therefore, it can be envisioned for surface regions having projecting filamentary elements to be spaced apart from each other on the surface of the braided structure.

Further preferably, the projecting filamentary elements form a so-called “bidirectional” arrangement on the filamentary braid. A bidirectional arrangement here is to be understood as meaning an arrangement in which the filamentary elements are oriented in two different directions. Considered preferentially in the longitudinal direction of the filamentary braid, it is preferable for the filamentary elements of a first filamentary-braid portion to be formed in the direction of a remaining second filamentary-braid portion and for the filamentary elements of the remaining second filamentary-braid portion to be formed in the direction of the first filamentary-braid portion. Considered preferentially in the longitudinal direction of the filamentary braid, it is particularly preferable for the filamentary elements of a first filamentary-braid portion to be oriented in the direction of the middle of the filamentary braid and for the filamentary elements of a remaining second filamentary-braid portion to be similarly oriented in the direction of the middle of the filamentary braid. The length of the filamentary-braid portions preferably is equal to about half the length of the filamentary braid. The filamentary-braid portions may comprise peripheral, areal or longitudinal portions of the filamentary braid.

The projecting filamentary elements advantageously have a certain minimum separation from each other. More particularly, filamentary elements, which are preferably formed from one filament, maintain a specific minimum separation from each other. As a result, it is particularly advantageous for the filament to be held sufficiently firmly, for example, by friction and/or pinching, in the filamentary braid and hence is not readily pulled out of the filamentary braid. Preferably, the projecting filamentary elements have a separation of 0.2 to 10 mm, in particular 0.5 to 5 mm, preferably 0.5 to 3 mm, relative to each other, measured from the exit points of the filamentary elements from the filamentary braid, preferably in the longitudinal direction of the suture. Filamentary elements formed from one filament can also have a larger separation from each other.

The filamentary braid can be formed from mono- and/or multifilaments. A combination of monofilaments and multifilaments is preferred, since the use of multifilaments renders the filamentary braid all together more supple and, more particularly, more flexible compared with a braided structure formed from monofilaments. Furthermore, a filamentary braid comprising multifilaments generally has a diminished or no memory effect.

The filaments having the projecting filamentary elements may account for a proportion of 2 to 80%, preferably 2 to 50%, more particularly 5 to 35%, more particularly 10 to 15%, based on the total number of filaments in the filamentary braid. The filamentary elements preferably form an angle α<90°, more particularly between 5 and 70°, with the surface of the filamentary braid.

In one form, the filamentary braid includes multiplied filaments (filaments taken in multiple ply in the braiding operation), more particularly doubled and/or tripled filaments. The multiplied filaments typically include mono- and/or multifilaments. The multiplied filaments may themselves include braided structures, more particularly braided multifilaments. Preferably, the multiplied filaments include at least one monofilament. The projecting filamentary elements can in principle come from multiplied filaments, more particularly doubled and/or tripled filaments. Preferably, the projecting filamentary elements comprise single filaments, more particularly single monofilaments, coming from multiplied filaments, more particularly doubled and/or tripled filaments. The filamentary braid itself can be formed for example from two single-ply filaments and one doubled filament and/or from three doubled filaments and two single filaments. In general, however, the braid includes distinctly more filaments, more particularly single and/or multiplied filaments. The use of multiplied filaments has the advantage that at least one single filament of the multiplied filament can be continuously involved in constructing the braided structure, while the remaining single filaments of the multiplied filament can be led out of the braided structure at identical or different points. This also has advantageous repercussions for the textile properties, such as the linear tensile strength for example, of the suture.

The filamentary elements projecting from the braided structure preferably come from stiffer, more particularly flexurally stiff, thicker or bulkier filaments than the other filaments of the filamentary braid. A higher flexural stiffness can be occasioned more particularly through a monofil structure on the part of the projecting filamentary elements. It is therefore preferable for the projecting filamentary elements to come from monofilaments of the filamentary braid. The projecting filamentary elements preferably come from monofilaments of the filamentary braid. Monofilaments have in principle a higher stiffness than multifilaments. Therefore, it can be envisioned that the stiff properties of monofilaments are exploited for a secure anchoring of the suture in a biological tissue. It can further be envisioned that the filaments having the projecting filamentary elements are monofilaments while the other filaments of the filamentary braid are multifilaments. Furthermore, higher stiffness, more particularly flexural stiffness, on the part of the projecting filamentary elements can also be achieved through a greater diameter, a higher intrinsic flexural stiffness (flexural modulus) and/or a higher hardness of the filamentary material from which the filamentary elements are produced.

It can further be preferable for the filamentary braid to include filaments of differing linear density. Preferably, the filaments whose filamentary elements project from the filamentary braid have a higher linear density than the remaining (other) filaments of the filamentary braid. The remaining/other filaments is here to be understood as referring to the filaments which are continuously involved in the construction of the braided structure. A higher linear density in this connection is to be understood as a greater ratio of filamentary mass to filamentary length. Given the same filamentary density, this further implies a higher ratio of filamentary diameter to filamentary length. The projecting filamentary elements preferably come from filaments having a linear density between 10 and 2500 dtex, more particularly 10 and 1700 dtex, preferably 20 and 700 dtex.

It can further be envisioned for the filamentary braid to include filaments of differing flexural stiffness. Preferably, the filaments whose filamentary elements project from the filamentary braid have a higher flexural stiffness than the remaining (other) filaments of the filamentary braid. The higher flexural stiffness can be due to a higher linear density on the part of the projecting filamentary elements. Alternatively or in combination thereto, however, the flexural stiffness of the projecting filamentary elements can also be increased by a subsequent reinforcement of the filamentary elements, more particularly by welding or by a chemical or physical treatment. This can be particularly advantageous in that it additionally improves the fixation of the suture in a biological tissue. The projecting filamentary elements preferably come from filaments having a flexural stiffness between 10 and 600 mN, more particularly 5 and 560 mN.

The projecting filamentary elements preferably have a length between 0.05 and 3 mm, more particularly 0.05 mm and 2 mm, preferably 0.20 and 1.5 mm. More particularly, the filaments whose filamentary elements project from the filamentary braid can have a diameter between 30 and 250 μm, more particularly 70 and 150 μm. The filamentary elements in question can have a round, oval, triangular, square, trapezoidal, rhomboid, pentagonal/five-cornered, hexagonal/six-cornered, star-shaped or cruciform cross section.

In principle, any biocompatible material is possible for producing the suture. The materials may comprise polymers, more particularly co- and/or terpolymers. The materials may also be present as block polymers, more particularly block copolymers and/or block terpolymers.

In another form, the suture is formed from nonabsorbable polymers, more particularly from polyurethanes, polyesters, polyamides, polyolefins, copolymers thereof, terpolymers thereof and/or mixtures thereof. Polyethylene terephthalate in particular is a suitable polyester. Polypropylene is one example of a possible polyolefin. The polyolefins contemplated may further be in a halogenated state. For example, the polyolefins may also comprise polyvinylidene difluoride (PVDF) and/or polytetrafluoroethylene, more particularly expanded polytetrafluoroethylene. Examples of possible polyamides are nylon-6,6 or nylon-6.

The suture may further be formed from absorbable polymers. Examples of suitable absorbable polymers are more particularly polyglycolide, polylactide, poly-ε-caprolactone, polytrimethylene carbonate, poly-p-dioxanone, 4-polyhydroxybutyric acid and/or mixtures thereof. Useful absorbable polymers may further comprise co- or terpolymers, more particularly block co- and/or block terpolymers, comprising at least one monomer from the group consisting of glycolide, lactide, ε-caprolactone, trimethylene carbonate, para-dioxanone and 4-polyhydroxybutyric acid.

Useful materials for producing the suture include more particularly the sutures commercially available from the applicant company under the names of Monosyn®, MonoPlus®, Dafilon®, Premilene®, and/or MonoMax®. Monosyn® is a synthetic monofil suture composed of polyglycolide or a copolymer of glycolide and lactide. Dafilon® is a nonabsorbable monofil suture composed of nylon-6 or nylon-6,6. Premilene® is a nonabsorbable monofil suture composed of polypropylene. MonoPlus® is a long-term absorbable monofil suture composed of polydioxanone. MonoMax® is an extremely long-term absorbable monofilament composed of 4-polyhydroxybutyric acid.

The suture may have a typical suture size, more particularly at least one suture size from the group consisting of USP 8/0, USP 7/0, USP 6/0, USP 5/0, USP 4/0, USP 3/0, USP 2/0, USP 0, USP 1, USP 2, USP 3, USP 4, USP 5 and USP 6.

The suture may be formed from absorbable and nonabsorbable materials. The suture may include both absorbable and nonabsorbable filaments. In the case of multiplied filaments, these can consist of absorbable and nonabsorbable single filaments. The projecting filamentary elements may be formed from an absorbable and a nonabsorbable material with the absorbable material preferably surrounding the nonabsorbable material, or vice versa (filaments having a sheath-core construction, more particularly biocomponent filaments). With regard to possible materials, reference is made to the description heretofore.

The filamentary braid may in principle include bioactive substances. The filamentary braid preferably includes growth factors, antiinflammatory compounds, analgesic substances and/or antimicrobials. The antimicrobials may comprise antimicrobial, more particularly antibacterial, compositions or compounds. For example, the antimicrobials may comprise anti-microbial metals, metal alloys or metal salts. Silver, copper, zinc and/or gold are in principle useful as antimicrobial metals. Preferably, the antimicrobial is silver or a silver salt. Anti-microbial metals and/or salts thereof, for example oxides, can be present in the form of nanoparticles and/or microparticles. Further possible antimicrobials include for example triclosan, chlorhexidine and/or polyhexamethylenebiguanide.

The filamentary braid may be a flat braid. In this form, the filamentary braid is normally formed from an odd number of strands. The filamentary braid can be tape-shaped in particular. The filamentary braid may also be a flat braid where the projecting filamentary elements are only disposed on one side, more particularly a face side, of the flat braid. It is additionally possible for the projecting filamentary elements to be disposed on both sides, more particularly both face sides, of a flat braid. Furthermore, the filamentary braid can be a flat braid where the projecting filamentary elements are only disposed at the edges of the flat braid. Combinations are also possible. With regard to further features and details, more particularly in respect of possible arrangements and orientations of the filamentary elements on the filamentary braid, the description heretofore is incorporated in full by reference.

Preferably, the filamentary braid is a round or tubular braid. In this form, the filamentary braid is typically formed from an even number of strands. The round or tubular braid preferably includes a core. The core itself can consist of an absorbable material. With regard to further properties of the core, more particularly concerning the material from which the core may be produced, the description heretofore is incorporated by reference. The description heretofore is also incorporated by reference concerning, for example, possible arrangements and orientations of the filamentary elements on the filamentary braid.

We further provide a process for producing the suture which comprises producing the filamentary braid by braidingly intertwining filaments to form a braided structure and forming filamentary elements projecting from the braided structure by leading individual filaments during braiding out of the braided structure which forms. As mentioned above, the individual filaments may comprise filaments of the braided structure, for example, single strands of multiplied filaments, and/or filaments of a core of the braided structure.

Advantageously, the number of filaments is kept substantially constant along the length of the suture. Preferably, the filaments which have been led out are led back into the braided structure and/or other filaments are led into the braided structure to keep the number of filaments substantially constant. It is more particularly envisioned for after every leading out of a filament a filament, more particularly a new filament and/or the current filament, to be inserted into the braided structure. In this way, an increasing tapering of the braided structure can be avoided.

Further preferably, the filaments which have been led out are cut off to form the filamentary elements projecting from the braid. In a more advanced form, the filamentary braid is produced by braidingly interlacing filaments to form a braided structure, leading individual filaments during the braiding operation out of the braided structure which forms and cutting them off to form the projecting filamentary elements, and also braiding new filaments into the braided structure. Preferably, the filaments which have been led out are cut off to a length between 0.05 and 3 mm, more particularly 0.05 and 2 mm and preferably 0.20 and 1.5 mm.

The filaments which have been led out may be led back into the braided structure with the formation of loops, more particularly in the manner of floats, overfeeds and/or velour loops. In this form, the filamentary loops formed can serve as anchoring structures of the suture. The filamentary loops formed may be opened, preferably severed, to form the projecting filamentary elements. The forms described in this section have the advantage of making it possible to produce the suture by continuous braiding. The loops can be favored by overfeeding the respective filament. In the case of multiplied filaments, then, at least one single filament can be overfed.

The opening of the filamentary loops can in principle be effected centrally or at a position deviating therefrom. Accordingly, it is possible to produce two filamentary elements projecting from the filamentary braid per loop which project from the filamentary braid either to equal length or to different lengths. It can be more particularly envisioned for one of these filamentary elements in each case to be cut off very close above the braided structure. The other filamentary element then has an orientation in one direction. In this way, it is possible to produce filamentary braids whose projecting filamentary elements point in different directions.

The projecting filamentary elements may be reinforced, more particularly stiffened. The reinforcement of the filamentary elements can be effected subsequently, i.e., after production of the braided structure. The filamentary elements can be, for example, welded, more particularly ultrawelded. A chemical reinforcement, more particularly consolidation, can likewise be carried out and can be effected, for example, by means of a coating with polymers. The measures described in this section are a particularly advantageous way to increase the flexural stiffness of the filamentary elements.

The filamentary elements may further be subjected to a subsequent orientation, i.e., after production of the braided structure. This subsequent orientation may comprise a purely mechanical orientation or an orientation that is thermal, i.e., brought about as a result of the action of heat.

Preferably, the filaments are taken at least doubled in the braiding operation. In this way, any impairment of the braided structure, more particularly in the course of the opening of filamentary loops described in the previous sections, can be largely avoided.

At least one end of the suture may be armed with a surgical needle. To arm a suture with a surgical needle, the suture is generally inserted into a dedicated hole in the needle and the needle is subsequently pressed together in the region of the hole.

We also provide a surgical kit comprising the suture and at least a surgical needle. With regard to further features and details concerning the kit, reference is made to the description heretofore.

We also provide for the use of the suture as a self-fixing or knotless suture. The suture is useful in particular for indications where the cosmetic result is of particular importance to the patient. Therefore, a further aspect relates to the use of the suture in plastic surgery and/or reconstructive surgery, more particularly for skin closure. In the field of plastic surgery, the suture is useful for face lifting and/or eyebrow lifting for example. The suture can be used more particularly for intracutaneous, subcutaneous or superficial skin closure.

The suture is further useful for wound regions which make it difficult or even impossible to knot conventional sutures. For example, the suture can be used in abdominal, gynecological and/or urological surgery. Further fields of application concern micro, eye, neuro, vascular, cardiac, abdominal and intestinal surgery. The suture is further suitable for use in endoscopic and/or laparoscopic surgery. The suture is also useful for closing trocar incisions, more particularly with so-called alpha stitches. The suture is further also useful for closing internal wounds.

A further aspect finally relates to the use of the suture for fixing implants, more particularly hernia meshes, preferably hernia meshes in the peritoneal region. In addition, it is also possible in principle to fix other implants, for example prolapse meshes and/or urinary incontinence meshes, by means of the suture.

Further features and details will be apparent from the following examples and figures. All the figures are hereby made part of this description by express reference. Individual features can each be actualized alone or two or more at a time in combination with each other.

### EXAMPLES

### Example 1

Two individual monofilaments composed of medical grade polypropylene and a doubled filament composed of medical grade polypropylene of USP size 5/0 are strand-braided together. Every ten interlacements one filament of the doubled filament is led out of the braid and led back into the braid with overfeeding. The loops projecting from the braid are subsequently opened, under a microscope using a cutting device with a blade, at a length corresponding to about 5/6 of the loop length. Subsequently, the short end of the opened loop is thermally fixed.

### Example 2

Three doubled filaments composed of medical grade polypropylene monofilaments of USP size 6/0 are strand-braided together. After every sixth interlacement one polypropylene monofilament is led out of the braid, overfed and led back into the braid. The loops are opened as in example 1. The short ends of the opened loops are subsequently thermally fixed. The single filaments led out always come from doubled filaments which alternate in sequence. In this way, only every third led-out filamentary end comes from one and the same doubled filament (spacing: 18 interlacements).

### Example 3

One tripled filament composed of medical grade polypropylene filaments and two single filaments of USP size 6/0 are strand-braided together. Every ten interlacements first the first single filament of the tripled filament is led out of the filamentary braid and is opened and fixed according to example 1. After a further interlacement the second single filament of the tripled filament is led out of the braid and fixed. In this way, the led-out filamentary ends occur in pairs along the length of the suture.

### Example 4

Three doubled filaments consisting of a medical grade polypropylene monofilament of USP size 6/0 and one braided multifilament composed of medical grade polyethylene terephthalate are strand-braided together. As in example 2, the monofilament is led out of the braid, overfed and fixed every six interlacements. The monofilaments led out come from doubled filaments which alternate in sequence. In this way, a particularly supple suture is obtained.

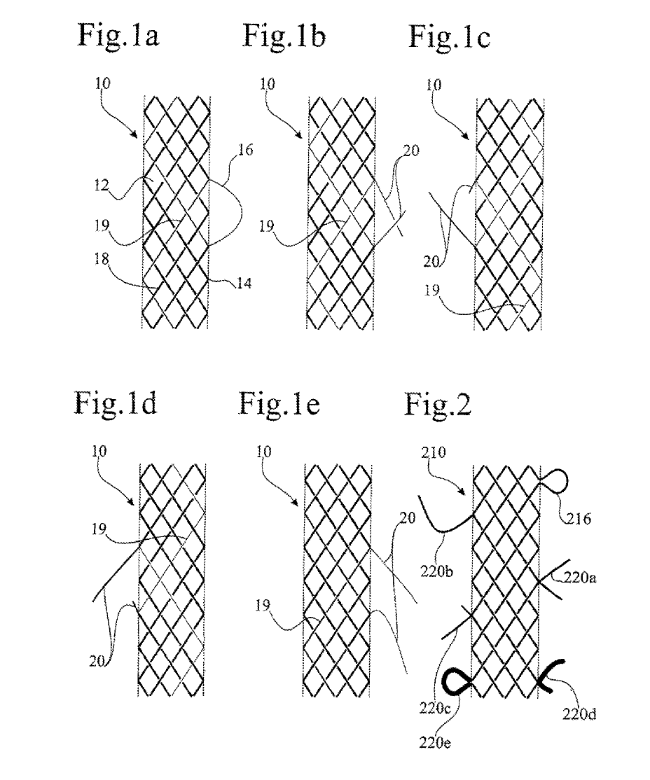

Turning now to the drawings, FIG. 1 is a schematic showing a detail from a suture 10 in the form of a filamentary braid 12 and also various forms of filamentary elements 16; 20 which protrude from the surface 14 of the filamentary braid 12. The filamentary braid 12 can be a round or flat braid. The filamentary braid 12 is braided from doubled filaments 18 (bold emphasis). The filamentary element 16 formed as a filamentary loop may comprise a float, overfeed or velour loop (FIG. 1a). The filamentary loop 16 may be produced by leading a single filament 19 out of the filamentary braid 12 and then reintroducing the single filament 19 back into the filamentary braid 12. The loop 16 may skip one braided strand 18 (FIG. 1a) or optionally two or more braided strands 18 before being incorporated back in the filamentary braid 12. Optionally, the loop 16 may also not skip any braided strand 18; that is, a led-out single filament 19 is directly inserted back into the filamentary braid 12. The loop 16 serves with particular advantage as anchoring structure for anchoring the suture 10 in a biological tissue.

However, it can also be envisioned to open the loop 16 to produce filamentary elements projecting from the filamentary braid 12 in the form of filamentary ends 20 (FIGS. 1b to 1e). On traction in the opposite direction, the filamentary ends 20 deploy and thereby anchor the suture 10 in the tissue. The opening of the loops 16 can in principle be effected in various ways. The loops 16 can be opened approximately centrally for example. This produces two filamentary ends 20 projecting from the filamentary braid 12, which generally each point in opposite directions (FIG. 1b). Normally, one of the two filamentary ends 20 is cut off or capped very closely above the filamentary braid 12. The cut-off or capped filamentary ends are optionally fixed that they may not detach from the braid. The direction of the projecting filamentary ends 20 is more particularly determined by the braiding angle and the length of the filamentary ends 20. This opens up the possibility of forming, in the longitudinal direction of the filamentary braid 12, filamentary ends 20 projecting from the filamentary braid 12 in opposite directions (FIGS. 1c and 1d). However, it is also possible to leave both of the filamentary ends 20 produced by opening one loop 16 standing as anchoring structures of the suture 10 (FIG. 1e). The two filamentary ends 20, which are each formed by the opening of one loop 16, can optionally also be of differing length. This depends on whether the loop 16 is opened centrally or at a position deviating therefrom. The embodiments described in FIGS. 1a-e in respect of the filamentary elements can also be applied, mutatis mutandis, to a filamentary braid composed of single filaments or to a filamentary braid composed of single and/or multiplied filaments, more particularly doubled and/or tripled filaments.

FIG. 2 is a schematic showing further possible ways of varying filamentary elements 216; 220a-e of a suture 210 which project from a braided structure 210. The filamentary elements 216; 220a-e projecting or protruding from the braided structure 210 can be stiffened in an additional step following production of the filamentary braid. Stiffening can be effected for example by means of welding or by coating with a polymer (welded or coated filamentary ends 220d and welded or coated loops 220e).

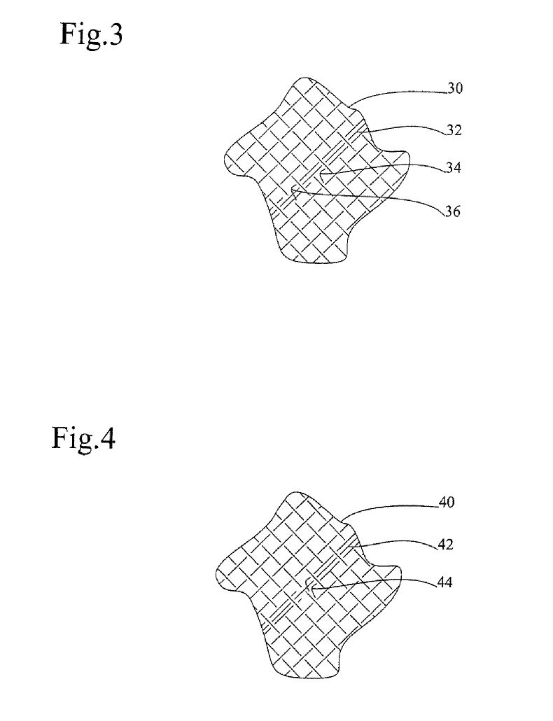

FIG. 3 is a schematic showing a plan view of a suture 30 having tripled filaments 32 (bundled filaments each with three filaments). Two single filaments of the tripled filament 32 lead out of the braid at different points and are formed as protruding filamentary ends 34 and 36.

FIG. 4 is a schematic showing a plan view of a suture 40 having tripled filaments 42 where two single filaments of the tripled filament 42 lead out of the braided structure at the same point. The single filaments led out are likewise formed as protruding filamentary ends 44.

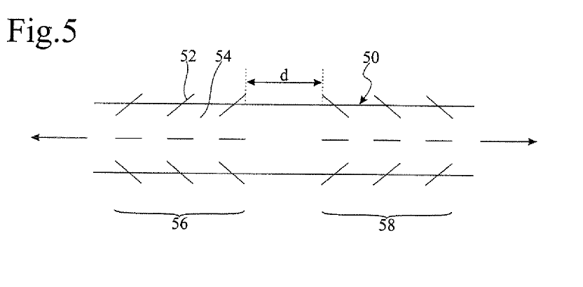

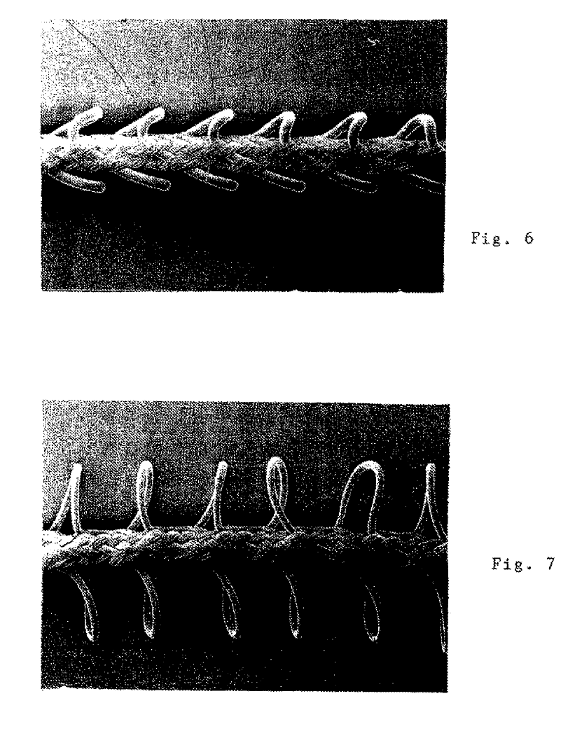

FIG. 5 is a schematic showing a side view of a suture 50, the filamentary elements 52 of which protrude from the braided structure in the form of filamentary ends. The filamentary ends 52 point for a first facial or peripheral portion 56 in the direction of the middle of the braided structure and for another second facial or peripheral portion 58 again in the direction of the middle of the braided structure. The facial or peripheral portions 56 and 58 can have a certain separation d from each other. The ends of the suture 50 can also each be connected to a surgical needle.

The suture can simultaneously include different types of protruding filamentary elements. For example, the suture can include both filamentary loops and filamentary ends that project from the braided structure. The filamentary elements can also be in different lengths and size ratios.

1. A surgical suture comprising a filamentary braid including filamentary elements projecting from the filamentary braid for anchoring in biological tissues.

2. The surgical suture according to claim 1, wherein the projecting filamentary elements come from filaments of the filamentary braid and/or from filaments of a core of the filamentary braid.

3. The surgical suture according to claim 1, wherein the projecting filamentary elements comprise ends of the filamentary braid and/or of a core of the filamentary braid, and/or filamentary loops.

4. The surgical suture according to claim 3, wherein the filamentary loops comprise floats, overfeeds and/or velour loops.

5. The surgical suture according to claim 1, wherein the projecting filamentary elements comprise opened filamentary opened floats, opened overfeeds and/or opened velour loops.

6. The surgical suture according to claim 1, wherein the projecting filamentary elements are in a reinforced state.

7. The surgical suture according to claim 1, wherein the projecting filamentary elements have a separation of 0.2 to 10 mm relative to each other measured From exit points of the filamentary elements from the filamentary braid, in the longitudinal direction of the suture.

8. The surgical suture according to claim 1, wherein the filaments having the projecting filamentary elements account for a proportion of 2 to 50%, based on the total number of filaments in the filamentary braid.

9. The surgical suture according to any claim 1, wherein the filamentary braid is formed from mono- and/or multifilaments.

10. The surgical suture according to claim 1, wherein the filamentary braid includes multiplied filaments.

11. The surgical suture according to claim 1, wherein the projecting filamentary elements comprise single filaments coming from multiplied filaments.

12. The surgical suture according to claim 1, wherein the filaments whose elements project from the filamentary braid have a higher flexural stiffness than remaining filaments of the filamentary braid.

13. The surgical suture according to claim 1, wherein the projecting filamentary elements come from filaments having a flexural stiffness between 10 and 600 mN.

14. The surgical suture according to claim 1, wherein the projecting filamentary elements come from monofilaments of the filamentary braid.

15. The surgical suture according to claim 1, wherein the filaments whose elements project from the filamentary braid have a higher linear density than remaining filaments of the filamentary braid.

16. The surgical suture according to claim 1, wherein the projecting filamentary elements come from filaments having a linear density between 10 and 2500 dtex.

17. The surgical suture according to claim 1, wherein the projecting filamentary elements have a length between 0.05 and 3 mm.

18. The surgical suture according to claim 1, wherein the projecting filamentary elements have a diameter between 30 and 250 μm.

19. The surgical suture according to claim 1, wherein the filamentary braid is a flat braid.

20. The surgical suture according to claim 1, wherein the filamentary braid is a round or tubular braid which includes a core.

21. A process for producing a suture according to claim 1, comprising:
producing the filamentary braid by braidingly intertwining filaments to form a braided structure; and
forming filamentary elements projecting from the braided structure by leading individual filaments during braiding out of the braided structure which forms.

22. The process according to claim 21, wherein the filaments which have been led out are led back into the braided structure and/or other filaments are led into the braided structure to keep the number of filaments substantially constant.

23. The process according to claim 21, wherein after every leading out of a filament a filament is inserted into the braided structure.

24. The process according to claim 21, wherein the filaments which have been led out are cut off, more particularly to a length between 0.05 and 3 mm.

25. The process according to claim 21, wherein the filaments which have been led out are led back into the braided structure with the formation of loops and, optionally, the loops are opened.

26. The process according to claim 21, wherein the filaments are taken at least doubled in the braiding operation.

27. A surgical kit comprising a suture according to claim 1 and at least a surgical needle.

28-30. (canceled)
